(19) United States
(12) Patent Application Publication
(10) Pub. No.: US 2005/0027193 A1
(43) Pub. Date: Feb. 3, 2005

(54) Method for automatically merging a 2D fluoroscopic C-arm image with a preoperative 3D image with one-time use of navigation markers

(76) Inventors: Matthias Mitschke, Nurnberg (DE); Norbert Rahn, Forchheim (DE); Dieter Ritter, Furth (DE)

Correspondence Address: SCHIFF HARDIN, LLP;PATENT DEPARTMENT, 6600 SEARS TOWER, CHICAGO, IL, 60606-6473, US

(21) Appl. No.: 10/851,259

(22) Filed: May 21, 2004

(30) Foreign Application Priority Data
May 21, 2003 (DE) 103 23 008.4

(51) Int. Cl. A61B 5/05

(52) U.S. Cl. 600/427

(57) ABSTRACT

In a method and apparatus for the automatic merging of 2D fluoroscopic C-arm images with preoperative 3D images with a one-time use of navigation markers, markers in a marker-containing preoperative 3D image are registered relative to a navigation system, a tool plate fixed on the C-arm system is registered in a reference position relative to the navigation system, a 2D C-arm image (2D fluoroscopic image) that contains the image of at least a medical instrument is obtained in an arbitrary C-arm position, a projection matrix for a 2D-3D merge is determined on the basis of the tool plate and the reference position relative to the navigation system, and the 2D fluoroscopic image is superimposed with the 3D image on the basis of the projection matrix.

### BACKGROUND OF THE INVENTION

1. Field of the Invention

The present invention concerns a method for superimposing or fusing a 2D image obtained with a C-arm x-ray system, with a preoperative 3D image. The invention particularly concerns the display of a medical instrument in the 3D image which is in the examination region of a patient and is included in the 2D image.

2. Description of the Prior Art

An increasing number of examinations or treatments of patients are performed minimally invasively, that is, with the least possible surgical trauma. Examples are treatments with endoscopes, laparoscopes, or catheters, all of which are inserted into the examination zone of a patient through a small opening in the body. Catheters, for example, are often used in the course of cardiological examinations.

A problem from a medical-technical point of view is that the medical instrument (in the following, a catheter will be referred to as a non-restrictive example) during the procedure (operation or examination) can be visualized very exactly and with high resolution using an intraoperative X-ray control with the C-arm system in one or more transirradiation images, also known as 2D fluoroscopic images, but the anatomy of the patient can be only insufficiently visualized in the 2D fluoroscopic images during the intervention. Moreover, the physician often has the desire within the scope of operation planning to display the medical instrument in a 3D image (3D dataset) obtained before the intervention (preoperatively.)

### SUMMARY OF THE INVENTION

An object of the present invention is to merge an intraoperatively obtained 2D fluoroscopic image showing the medical instrument in a simple way with a preoperatively obtained 3D image.

This object is solved in accordance with the invention by a method for automatic merging of a 2D fluoroscopic C-arm image with a preoperative 3D image using navigation markers wherein markers in a marker-displaying preoperative 3D image are registered relative to a navigation system, a tool plate fixed to a C-arm system is registered in a reference position relative to the navigation system, a 2D C-arm image (2D fluoroscopic image) that contains the image of at least a medical instrument in an arbitrary C-arm position is obtained, a projection matrix for a 2D/3D merge is determined on the basis of the tool plate and reference positions relative to the navigation system, and the 2D fluoroscopic image is superimposed onto the 3D image E on the basis of the projection matrix.

The preoperative 3D image containing the markers can be a pre-existing image that is stored and made available to a computer wherein the automatic merging takes place.

In a first alternative embodiment, artificial markers are used and the preoperative 3D image containing the artificial markers is obtained after the artificial markers have been set relative to the patient. This can ensue, if necessary, by surgically opening the patient or, if suitable, the artificial markers can be fixed on the surface of the body. After the artificial markers are set in one of these ways, registration of the set of artificial markers is then undertaken.

In a second alternative embodiment of the method of the invention, anatomical markers are used, which are identified and registered.

Ideally, the reference position is measured with a fixed chassis, 0° angulation, and 0° orbital angle of the C-arm.

The preoperative 3D image can be obtained in different ways, for instance using magnetic resonance tomography, computed tomography, ultrasound, positron emission tomography, or nuclear medicine.

The above object also is achieved in accordance with the principles of the present invention in a C-arm x-ray imaging device for implementing the above-described method.

### DESCRIPTION OF THE DRAWINGS

FIG. 1 is a schematic illustration of a medical examination and/or treatment system in accordance with the invention.

FIG. 2 is an illustration for explaining a marker-based registration of a 3D image with a 2D fluoroscopic image in accordance with the invention.

FIG. 3A is a flowchart of the inventive method, using artificial markers.

FIG. 3B is a flowchart of the inventive method, using anatomical markers.

### DESCRIPTION OF THE PREFERRED EMBODIMENTS

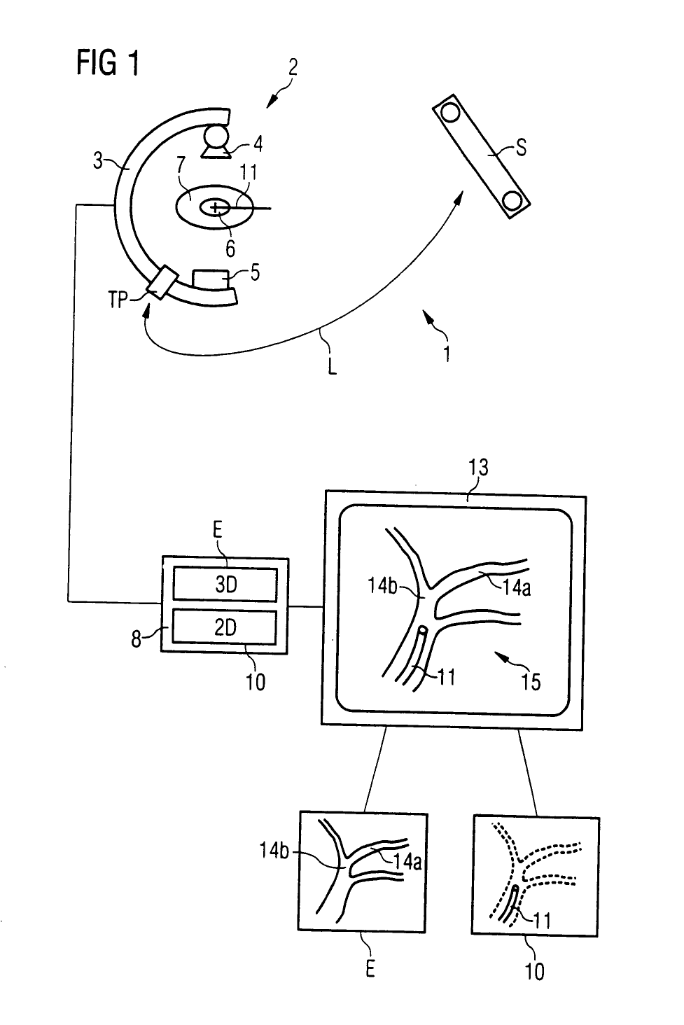

FIG. 1 schematically illustrates an examination and/or treatment system 1 in accordance with the invention, with only basic components being shown. The system includes an imaging system 2 to obtain two-dimensional transillumination images (2D fluoroscopic images). The imaging system 2 has a C-arm 3, to which an X-ray radiation source 4, and a radiation detector 5, for instance a solid body imaging detector, and a tool plate TP are attached. The examination zone 6 of a patient 7 is located ideally in the isocenter of the C-arm 3, so that its entire extent is visible in the captured 2D fluoroscopic image.

In the immediate vicinity of the imaging system 2, there is a navigation sensor S, by means of which the current position of the tool plate TP can be recorded and thus the C-arm 3, as well as the position and orientation of a medical instrument 11 used for the procedure, and the patient.

The system 1 is operated using a control and processing unit 8, which among other things controls the image data acquisition. It also includes an image processing unit, not shown in detail. In this unit, among other things, is a 3D image data set E, which ideally is recorded preoperatively. This preoperative data set E can be recorded with any arbitrary imaging modality, for example with a computed tomography device CT, a magnetic resonance tomography device MRT, an ultrasound device UR, a nuclear medicine device NM, a positron emission tomography device PET, etc. The data set E alternatively can be recorded as a quasi intraoperative data set with its own imaging system 2, thus directly before the actual intervention, whereby the imaging system 2 would then be operated in a 3D angiography mode.

In the example shown, a catheter 11 is introduced into the examination zone 6, here the heart. The position and orientation of this catheter 11 can first be detected using the navigation system S, and then visualized with an intraoperative C-arm image (2D fluoroscopic image) 10. Such an image is shown in FIG. 1 as an enlarged conceptual sketch.

The current invention provides a method in which an intraoperative 2D fluoroscopic image 10 recorded in an arbitrary C-arm position, which includes the medical instrument 11 (here a catheter), is automatically, that is using a computer and the processing system 8, overlaid (merged) with the preoperative 3D image E, so that the visualization and navigation of the instrument in the 3D data set E is possible. The result of such a merge is shown in FIG. 1 in the form of an overlay image 15 displayed on a monitor 13.

In order to be able to obtain a correct (correctly oriented) overlay of intraoperative 2D fluoroscopic images with the preoperative 3D data set E, it is necessary to register both images relative to one another or each relative to the navigation sensor S. Registration of two image data sets (of three-dimensional and/or two-dimensional nature) means to correlate their coordinate systems with one another, or to derive a mapping process which converts one image data set into the other. In general, such a mapping process or registration is specified using a matrix. The term “matching” is often used for such a registration. Among other words for registration are “merging” or “correlation”. Such a registration, for instance, can be performed interactively by the user on a display screen.

There are different possibilities for the registration of the two images:
1. One possibility is to identify a reasonable number (at least two) of image elements in the 2D fluoroscopic image, identifying the same image element or elements in the 3D image, then reorienting this 3D image relative to the 2D fluoroscopic image through translation and/or rotation and/or 2D projection. This type of image elements are called “markers” and can be anatomical in origin or also artificially attached.

Markers of anatomical origin—such as for instance blood vessel branching points or small sections of coronary artery, but also the corner of the mouth or the tip of the nose—are called “anatomical markers”. Artificially inserted or attached marking points are called “artificial markers”. Artificial markers are, for instance, screws which are set in a preoperative procedure, or simply objects which are attached to the surface of the body (for instance, glued in place).

Anatomic or artificial markers can be determined interactively by the user in the 2D fluoroscopic image (for instance, by clicking on the display) and then searched for and identified in the 3D image using suitable analysis algorithms. Such a registration is called “marker-based registration”.

2. A second possibility is so-called “image-based registration”. Here, a 2D project image is created from the 3D image in the form of a digitally reconstructed radiogram (DRR), which is compared to the 2D fluoroscopic image with regards to its matching features, whereby, to optimize the comparison, the DRR image is changed using translation and/or rotation and/or stretching relative to the 2D fluoroscopic image, until the matching features of both images have reached a given minimum. It is practical for the user to move the DRR image after its creation into a position in which it is as similar as possible to the 2D fluoroscopic image and only then to initiate the optimization cycle, in order to minimize the processing time for the registration.

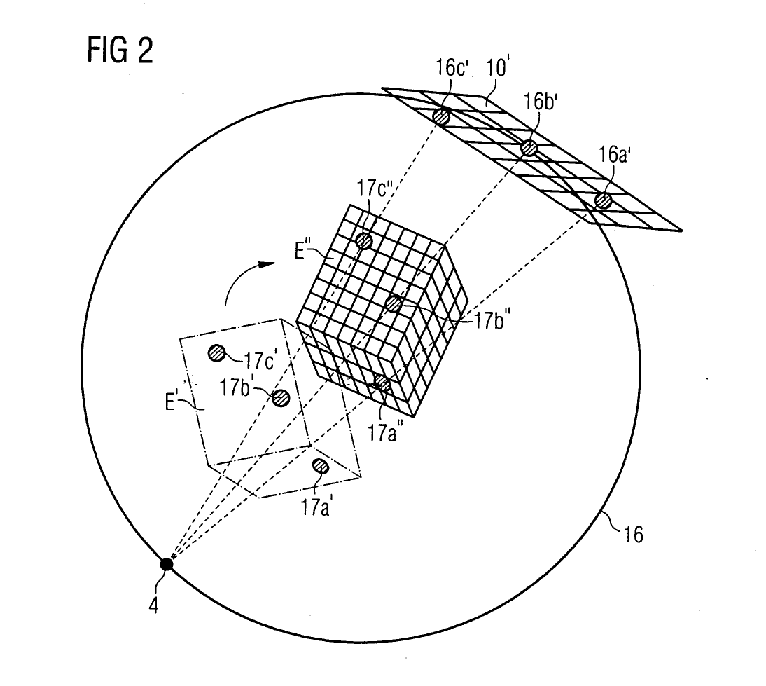

FIG. 2 is an illustration for explaining marker-based registration of a 3D image with a 2D fluoroscopic image. A 2D fluoroscopic image 10′ is shown which is recorded by a detector 5 in the same position, not shown. The radiation source 4 or its focus is also shown, along with the movement trajectory 16 of the C-arm, on which the detector 5 and the radiation source 4 are moved.

Also shown is the original 3D image E′ immediately after it is obtained, without it being registered relative to the 2D fluoroscopic image 10′.

For registration, there are identified or defined several markers—in the example shown, three spherical artificial markers 16a′, 16b′, and 16c′. These markers are also identified in the original 3D image E′. As can be seen from FIG. 2, the markers 17a′, 17b′, 17c′ are located in positions in the original 3D image in which they do not lie directly on the projection lines running from the radiation source 4 to markers 16a′, 16b′, 16c′ in the 2D fluoroscopic image. If the markers 17a′, 17b′, 17c′ were projected onto the detector plane, they would lie in clearly different positions than the markers 16a′, 16b′, 16c′.

For registration, the 3D image E′ is now moved through translation and rotation (in this example, no scaling is necessary) until the markers 17a″, 17b″, 17c″ of the repositioned 3D image E″ can be projected onto the markers 16a′, 16b′, 16c′, and the registration is now complete.

Both image-based and marker-based registration have significant disadvantage. A marker-based registration often makes an additional operative procedure necessary to set artificial markers. Anatomic markers are often difficult to locate uniquely, often making calibration relative to a marker-based registration error-prone. Image-based registration requires very long processing times and, due to numerical instabilities, is a very unreliable procedure and therefore seldom used.

The identification of markers in marker-based registration need not necessarily be performed on the display screen. If a navigation system is present (navigation sensor S, see FIG. 1) and in preparation for a navigation-supported intervention, a marker-based registration of a (for instance) preoperative 3D image relative to the navigation system S is performed by the physician via manual selection of artificial or anatomical markers with a navigation pointer. Since the medical instrument 11 is registered relative to the navigation system with respect to position and orientation due to existing detectors, such a correlation between the medical instrument 11 and the preoperative 3D image E is thus created. Using the control and processing unit 8, the current image of the medical instrument 11 thus can be integrated and visually merged into the 3D image. Navigation of the medical instrument in E is thus possible.

However, navigation-supported registration still presents significant disadvantages: if it is desired to register intraoperatively recorded 2D fluoroscopic images with the preoperative 3D image on a navigation-supported basis, then in a navigation-supported marker-based registration, the markers would have to be manually selected for each C-arm position of the 2D fluoroscopic image to be recorded. Such a procedure, in practice, is very error-prone and tedious. If the markers are selected in a different order in the image from those in the patient, anatomic markers cannot be found in a reproducible way, or if the relative orientation of the markers has changed, erroneous positioning will result. In addition, if navigation is misadjusted at any point during the intervention, registration must be repeated each time. In a conventional marker- or image-based registration, the above disadvantages apply to the corresponding procedure.

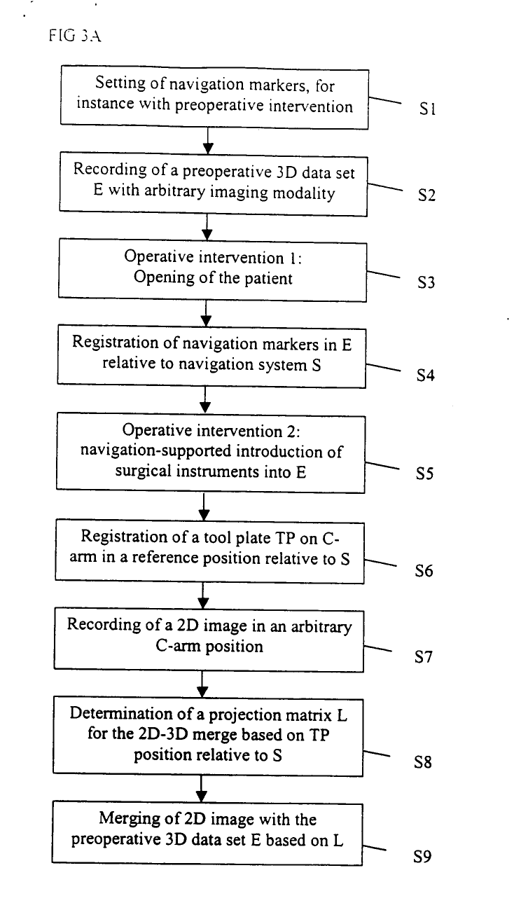

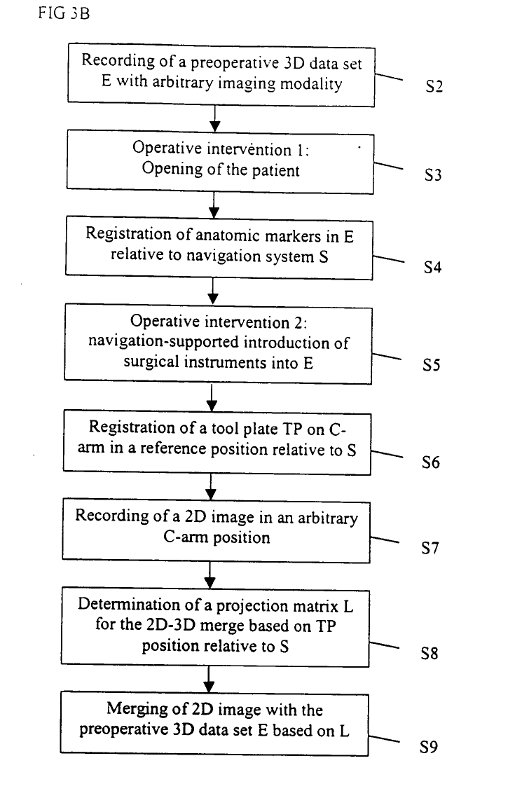

The method of the invention still uses navigation markers (navigation-supported or computer-based). However, to avoid or significantly decrease the disadvantages of a marker-based merge, in the method of the invention the problematic marker-based registration must be performed only for the first 2D fluoroscopic image to be merged, or an already existing marker-based registration from the navigation procedure for the medical instrument can be used. For all further 2D-3D merges required during the intervention or examination, no additional interactive registration is necessary, as will be shown using the process flowcharts in FIGS. 3A and 3B.

FIG. 3A is a schematic representation of the method of the current invention for automatic merging of 2D fluoroscopic images with preoperative 3D images with a one-time use of artificial markers. The method involves nine steps:

In a first step S1, artificial markers are set in a preoperative intervention. A preoperative intervention is not necessary if the artificial markers can, for example, be glued to the patient's skin. In a second step S2, a preoperative 3D data set E is recorded, in which all artificial markers are included and can be displayed. The 3D data set can be recorded with any arbitrary image capture modality (MRT, CT, PET, US, etc.) In a third step S3, a first operative intervention is performed in which the patient is opened, in order to register the artificial markers in E relative to a navigation system S in a fourth step S4. The registration is performed by manual selection of the markers with a navigation pointer. An operative intervention as in step S3 is not necessary if the markers are attached to the surface of the body (for instance, glued). In a fifth step, a second operative intervention is performed, in which a surgical instrument registered in S can be introduced with navigational support into E. In order to be able to merge arbitrary intraoperative 2D fluoroscopic images with E intraoperatively during such a navigation-supported operation, in step S6 a tool plate fixed on the C-arm is registered in system S in a reference position of the C-arm. If now a 2D fluoroscopic image is recorded in a seventh step S7 in an arbitrary C-arm position, this can be registered (merged) relative to E on the basis of knowledge of the current C-arm position during the recording. Thus in an eighth step S8, a projection matrix L is determined with which a 2D-3D image merge can be performed. In a final step S9, the 2D fluoroscopic image can finally be merged with the 3D image on the basis of L.

The projection matrix L is derived by measuring the position of the tool plate fixed on the C-arm in a defined C-arm position. This results in a tool plate reference position TP_{Ref}, which is for example measured with a fixed chassis, 0° angulation, and 0° orbital angle. Since both TP_{Ref} and E are known in S, the new position of the tool plate TP in any arbitrary C-arm position (defined relative to S through TP) can be calculated relative to S. The registration characterized by L is thus given by determination of TP relative to S and thus to E. L can be used to give the desired merge of the 2D fluoroscopic image with the preoperative 3D data directly.

FIG. 3B is a schematic representation of the same method of the invention as that shown in FIG. 3A, whereby the method in FIG. 3B shows a variant in which not artificial, but rather anatomic markers are used. This eliminates the setting of markers; the first step S1 of the method in FIG. 3a is eliminated. In step S4 of the variant method in FIG. 3B, not artificial markers but appropriate anatomic structures (anatomic markers) are identified and registered.

Using the invented method, the problems of marker-based registration (merging) are minimized. The method utilizes the navigation procedure required for a navigation-supported intervention, whereby the problematic registration is only performed for the first image to be merged.

It should also be noted, that for the determination of L at an angulation # 0°, a C-arm distortion can occur, which can be corrected using look-up tables. The determination of a position matrix for C-arm devices is sufficiently well-known and need not be explained in further detail.

Although modifications and changes may be suggested by those skilled in the art, it is the intention of the inventors to embody within the patent warranted hereon all changes and modifications as reasonably and properly come within the scope of their contribution to the art.

1. A method for automatically merging a 2D fluoroscopic image, obtained with a C-arm apparatus having a movable C-arm and having a tool plate fixed on the C-arm, with a preoperative 3D image, comprising the steps of:
before undertaking a medical interventional procedure on a patient involving interaction of a medical instrument with the patient, making a marker-containing preoperative 3D image, obtained prior to the medical intervention, available to a computer and, in the computer, automatically registering the markers in the preoperative 3D image relative to a navigation system;
registering the tool plate on the C-arm in a reference position relative to the navigation system;
without using markers, obtaining a 2D fluoroscopic image of a region of the patient in which said medical instrument is disposed with said C-arm in an arbitrary position;
in said computer, determining a projection matrix for merging said 2D fluoroscopic image and said preoperative 3D image dependent on said reference position and said projection matrix relative to the navigation system; and
merging said 2D fluoroscopic image with said preoperative 3D image using said projection matrix.

2. A method as claimed in claim 1 wherein the step of making said marker-containing preoperative 3D image available to said computer comprises making said maker-containing preoperative 3D image electronically available to said computer from a memory in which said marker-containing preoperative 3D image is stored as a pre-existing image.

3. A method as claimed in claim 1 comprising obtaining said marker-containing preoperative 3D image using said C-arm apparatus.

4. A method as claimed in claim 3 comprising employing artificial markers as said markers, and comprising setting said artificial markers relative to the patient prior to obtaining said marker-containing preoperative 3D image using said C-arm apparatus.

5. A method as claimed in claim 4 wherein the step of setting said artificial markers comprises surgically opening the patient and setting said artificial markers in the opened patient.

6. A method as claimed in claim 4 wherein the step of setting said artificial markers comprises fixing said artificial markers to a body surface of the patient.

7. A method as claimed in claim 3 comprising employing anatomical markers as said markers.

8. A method as claimed in claim 1 comprising obtaining said reference position of said tool plate with said C-arm apparatus in a fixed position with 0° angulation and 0° orbital angle of said C-arm.

9. A method as claimed in claim 1 comprising obtaining said marker-containing preoperative 3D image using an imaging modality selected from the group consisting of magnetic resonance tomography, computed tomography, ultrasound, positron emission tomography, and a nuclear medicine procedure, and storing said preoperative 3D image in a memory accessible by said computer.

10. EDIT An apparatus for automatically merging a 2D fluoroscopic image, obtained with a C-arm apparatus having a movable C-arm and having a tool plate fixed on the C-arm, with a preoperative 3D image, comprising the steps of:
before undertaking a medical interventional procedure on a patient involving interaction of a medical instrument with the patient, making a marker-containing preoperative 3D image, obtained prior to the medical intervention, available to a computer and, in the computer, automatically registering the markers in the preoperative 3D image relative to a navigation system;
registering the tool plate on the C-arm in a reference position relative to the navigation system;
without using markers, obtaining a 2D fluoroscopic image of a region of the patient in which said medical instrument is disposed with said C-arm in an arbitrary position;
in said computer, determining a projection matrix for merging said 2D fluoroscopic image and said preoperative 3D image dependent on said reference position and said projection matrix relative to the navigation system; and
merging said 2D fluoroscopic image with said preoperative 3D image using said projection matrix.
